(19) United States
(12) Patent Application Publication
(10) Pub. No.: US 2022/0059217 A1
(43) Pub. Date: Feb. 24, 2022

(54) INFORMATION PROCESSING APPARATUS, INFORMATION PROCESSING SYSTEM, AND NON-TRANSITORY STORAGE MEDIUM

(71) Applicant: Toyota Jidosha Kabushiki Kaisha, Toyota-shi Aichi-ken (JP)

(72) Inventors: Takaharu Ueno, Nagoya-shi (JP); Yu Nagata, Tokyo-to (JP); Yurika Tanaka, Yokosuka-shi (JP); Ryosuke Kobayashi, Nagakute-shi (JP); Shintaro Matsutani, Kariya-shi (JP); Syouta Komatsu, Toyota-shi (JP)

(21) Appl. No.: 17/404,353

(22) Filed: Aug. 17, 2021

(30) Foreign Application Priority Data
Aug. 19, 2020 (JP) 2020-138716

(51) Int. Cl. G16H 40/20; G16H 10/60

(52) U.S. Cl. CPC G16H 40/20; G16H 10/60

(57) ABSTRACT

An object of the present disclosure is to estimate a required time for medical care in relation to a user visiting a medical establishment. An information processing apparatus estimates the required time for medical care in relation to the user visiting the medical establishment. The information processing apparatus includes a controller configured to determine a scheduled medical examination item as at least one medical examination item that is scheduled for the user, calculate a first required time based on the scheduled medical examination item, receive a correction of the scheduled medical examination item while the user is receiving medical care, and calculate a second required time based on the scheduled medical examination item after the correction.

### CROSS REFERENCE TO THE RELATED APPLICATION

This application claims the benefit of Japanese Patent Application No. 2020-138716, filed on Aug. 19, 2020, which is hereby incorporated by reference herein in its entirety.

### BACKGROUND

### Technical Field

The present disclosure relates to a technology for supporting a user in a medical establishment.

### Description of the Related Art

At the time of a user visiting a medical establishment, there is a demand to predict an expected time required for medical care. Accordingly, for example,

Patent Literature 1 discloses a system that estimates a required time for medical care based on an electronic medical chart corresponding to the user and medical interview sheet data.

### CITATION LIST

### Patent Literature

Patent Literature 1: Japanese Patent Laid-Open No. 2019-185252

### SUMMARY

According to the conventional technology, a required time for medical care for each user and wait times of other users may be estimated.

However, a required time for medical care is difficult to be estimated with high accuracy just by referring to past medical records or contents of a medical interview sheet. For example, an unscheduled examination or treatment may occur as a result of a doctor actually performing medical care. Conversely, a scheduled examination may be decided to be unnecessary. In such cases, the following medical schedule may be moved forward or backward, possibly causing an increase in the wait time or occurrence of a vacant time.

An object of the present disclosure is to provide a technology for estimating a required time for medical care in relation to a user visiting a medical establishment.

A first mode of the present disclosure is an information processing apparatus for estimating a required time for medical care in relation to a user visiting a medical establishment.

More specifically, the information processing apparatus may include a controller configured to: determine a scheduled medical examination item as at least one medical examination item that is scheduled for the user; calculate a first required time based on the scheduled medical examination item; receive a correction of the scheduled medical examination item while the user is receiving medical care; and calculate a second required time based on the scheduled medical examination item after the correction.

Furthermore, a second mode of the present disclosure is an information processing system including a first apparatus configured to estimate a required time for medical care in relation to a user visiting a medical establishment, and a second apparatus that is associated with the user.

More specifically, in the information processing system, the first apparatus may include a controller configured to: determine a scheduled medical examination item as at least one medical examination item that is scheduled for the user; calculate a first required time based on the scheduled medical examination item; receive a correction of the scheduled medical examination item while the user is receiving medical care; calculate a second required time based on the scheduled medical examination item after the correction; create a medical schedule for a plurality of the users based on the first required time and the second required time; and transmit a predetermined notification to the second apparatus based on the medical schedule.

Furthermore, a third mode of the present disclosure is a non-transitory storage medium storing a program.

More specifically, the program may cause an information processing apparatus for estimating a required time for medical care in relation to a user visiting a medical establishment to: determine a scheduled medical examination item as at least one medical examination item that is scheduled for the user; calculate a first required time based on the scheduled medical examination item; receive a correction of the scheduled medical examination item while the user is receiving medical care; and calculate a second required time based on the scheduled medical examination item after the correction.

Furthermore, as another mode, there may be cited a method that is performed by the information processing apparatus described above, or a computer-readable storage medium non-transitorily storing the program described above.

According to the present disclosure, a required time for medical care may be estimated in relation to a user visiting a medical establishment.

### BRIEF DESCRIPTION OF THE DRAWINGS

FIG. 1 is a diagram for giving an overview of an information processing system according to a first embodiment;

FIG. 2 is a diagram illustrating, in detail, structural elements of the information processing system according to the first embodiment;

FIGS. 3A to 3C are diagrams for describing data that is stored in a storage;

FIGS. 4A and 4B are diagrams for describing a medical schedule;

FIG. 5 is a flow diagram illustrating a flow of data transmitted and received in a first phase;

FIG. 6 is a flow diagram illustrating a flow of data transmitted and received in a second phase;

FIG. 7 is a flowchart of a process that is performed by a server apparatus; and

FIG. 8 is a diagram for giving an overview of an information processing system according to a second embodiment.

### DESCRIPTION OF THE EMBODIMENTS

There is a system that estimates a time required for medical care based on information about a patient (hereinafter referred to as “user”) visiting a medical establishment. By estimating the time required for medical care for each person, medical care for a plurality of patients using the medical establishment may be scheduled.

However, a required time for medical care cannot always be predicted with accuracy just by referring to information that is given in advance. For example, if a cavity is found when a person visits a dentist for a regular check-up, a time for treatment may become necessary. Furthermore, if the cause of a pain is determined by a medical interview or palpation, a scheduled examination may become unnecessary. That is, a scheduled required time is possibly increased or decreased after medical care is started, thereby causing the following medical schedule to be moved forward or backward.

An information processing apparatus according to a present embodiment may cope with such a problem.

The information processing apparatus according to the present embodiment is an information processing apparatus that estimates a required time for medical care in relation to a user visiting a medical establishment. More specifically, there may be included a controller configured to: determine a scheduled medical examination item as at least one medical examination item that is scheduled for the user; calculate a first required time based on the scheduled medical examination item; receive a correction of the scheduled medical examination item while the user is receiving medical care; and calculate a second required time based on the scheduled medical examination item after the correction.

The scheduled medical examination item is a medical examination item that is scheduled in advance. For example, the scheduled medical examination item may be determined based on information that is known when a reservation is made for medical care, past medical records, medical interview sheet data and the like. Furthermore, the first required time is a required time that is calculated based on the scheduled medical examination item. The scheduled medical examination item is merely a medical examination item that is scheduled (such as one that is automatically estimated by an apparatus), and is possibly changed by judgement of a doctor or the like. Similarly, the first required time is also possibly changed.

The controller may receive a correction of the scheduled medical examination item while the user is receiving medical care. For example, the scheduled medical examination item is corrected in a case where there is a medical examination item that is decided to be unnecessary by a doctor or a nurse or there is a medical examination item that is newly decided to be necessary by a doctor or a nurse. Then, the second required time is calculated based on the scheduled medical examination item after the correction. The first required time is thus updated to the second required time.

According to such a configuration, the time required for medical care may be re-calculated when a doctor or the like starts providing medical care to the user. The following medical schedule may thus be adjusted or re-created swiftly, and convenience of other users may be increased. Moreover, an end time may be notified with high accuracy to the user receiving medical care.

Furthermore, the information processing apparatus may be characterized by further including a storage configured to store required time data that is data associating a required time with each medical examination item, where the controller calculates the first required time and the second required time based on the required time data.

Defining the required time for each medical examination item enables more accurate estimation of the required time.

Furthermore, the controller may be characterized by determining the scheduled medical examination item based on information that is obtained from a result of a preliminary medical interview with the user.

The medical interview may be performed in the form of an interview sheet, for example. The medical interview may be performed at the time of reservation for medical care, or may be performed after the user visits the medical establishment. The result of the medical interview may be acquired through an information terminal carried by the user, an information terminal provided at the medical establishment, or the like, for example.

Furthermore, the controller may be characterized by determining the scheduled medical examination item based on a medical record of the user.

The medical record is data recording a history of medical care and the like for the user in the medical establishment. The medical record may include an examination record, a surgical record, a nursing record, and the like. The medical record may be acquired from outside the apparatus.

Furthermore, the controller may be characterized by correcting the scheduled medical examination item based on information that is acquired from a healthcare worker who is providing medical care to the user.

According to such a configuration, the medical examination item may be corrected based on judgment of a doctor or the like actually providing medical care to the user.

Furthermore, the controller may be characterized by creating a medical schedule for a plurality of the users based on the required time for medical care estimated for the plurality of the users.

In a case where the required time for medical care is changed for a user, the medical schedule is desirably changed for other users to match such a change.

Furthermore, in a case where the second required time is calculated for a first user, the controller may be characterized by adjusting the medical schedule for a second user who is to receive medical care after the first user.

According to such a configuration, a delay from a scheduled start time of medical care and the like may be announced, for example.

Furthermore, the controller may be characterized by estimating, based on the medical schedule, a time of end of medical care for a predetermined user.

A time when medical care for a specific user ends may be estimated based on the medical schedule after adjustment.

Furthermore, the controller may be characterized by transmitting the estimated time to an apparatus related to the user. For example, the apparatus related to the user may be an apparatus that manages a mobile body that the user gets on after receiving medical care, or may be an apparatus that is associated with a pharmacy that provides medicine to the user.

By transmitting the estimated time (that is, the time of end of medical care) to such an apparatus, convenience of the user may be increased. For example, by transmitting the estimated time to a pharmacy, medicine may be prepared in a timely manner at the time of visit by the user. Moreover, by transmitting the estimated time to an operator managing mobile bodies (such as taxis and autonomous vehicles), the user may be picked up after medical care.

Hereinafter, embodiments of the present disclosure will be described with reference to the drawings. Configurations of the following embodiments are merely examples, and the present disclosure is not limited to the configurations of the embodiments.

### First Embodiment

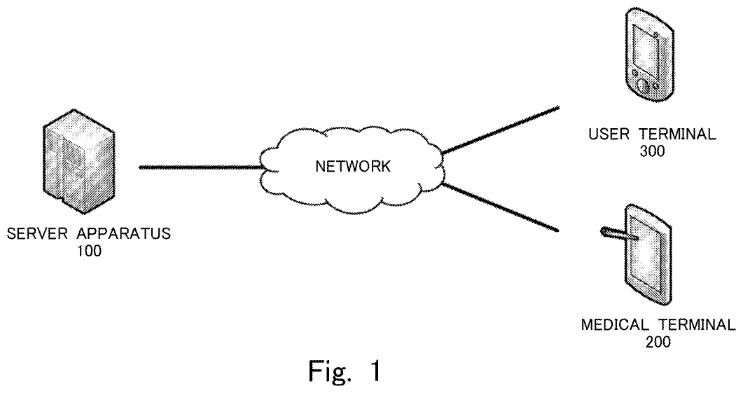

An overview of an information processing system according to a first embodiment will be given with reference to FIG. 1. The information processing system according to the present embodiment is configured to include a server apparatus 100 that creates a medical schedule for at least one user, a medical terminal 200 installed at a medical establishment, and a user terminal 300 carried by a user.

The server apparatus 100 is an apparatus that schedules medical care for a user at the medical establishment. The server apparatus 100 creates a medical schedule based on information transmitted from the medical terminal 200 and the user terminal 300 described later. The created medical schedule is updated as appropriate according to circumstances, and the result is transmitted to a related terminal.

Additionally, in the following description, creation of a medical schedule and scheduling of medical care each refer to a process of allotting a medical care time to each user.

The user terminal 300 is a computer that is used to apply for medical care. For example, the user terminal 300 may be a mobile terminal owned by the user. The user terminal 300 is capable of communicating with the server apparatus 100 over a network.

The medical terminal 200 is a computer that is installed at the medical establishment. The medical terminal 200 may be a dedicated terminal of healthcare workers, or may be a terminal that is shared by healthcare workers and users. The medical terminal 200 is capable of communicating with the server apparatus 100 over the network.

Additionally, in the example in FIG. 1, the server apparatus 100 is in one-to-one correspondence with each of the medical terminal 200 and the user terminal 300, but one server apparatus 100 may be in charge of a plurality of medical terminals 200 and a plurality of user terminals 300.

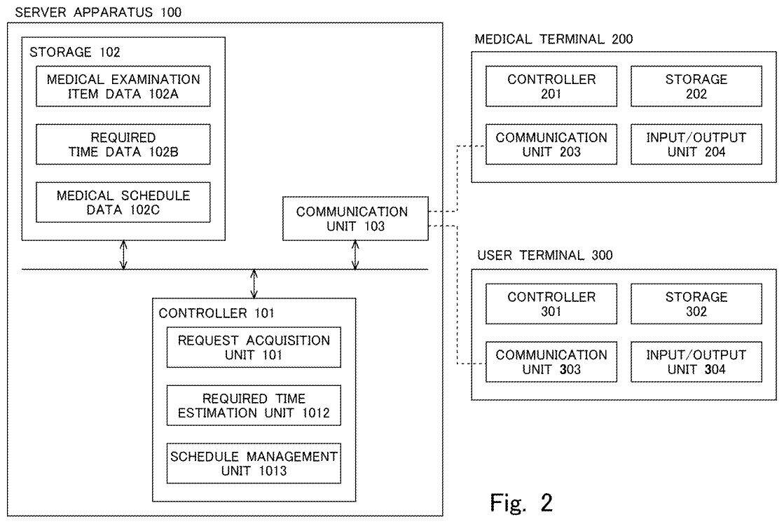

FIG. 2 is a diagram illustrating, in detail, structural elements of the information processing system according to the present embodiment. First, details of the server apparatus 100 will be given.

The server apparatus 100 schedules medical care for a plurality of users based on requests transmitted from a plurality of user terminals 300. Furthermore, the server apparatus 100 updates a created medical schedule based on information transmitted from the medical terminal 200.

The server apparatus 100 may be configured as a general-purpose computer. That is, the server apparatus 100 may be configured as a computer including processors such as a CPU and a GPU, main memories such as a RAM and a ROM, and auxiliary memories such as an EPROM, a hard disk drive and a removable medium. Additionally, the removable medium may be a USB memory, or a disk recording medium such as a CD or a DVD, for example. The auxiliary memory stores an operating system (OS), various programs, various tables and the like, and when a program stored in the auxiliary memory is loaded into a work area of the main memory and executed and each structural unit or the like is controlled through execution of the program, each function matching a predetermined object as described later may be implemented. However, one or some or all of functions may alternatively be implemented by a hardware circuit such as an ASIC or an FPGA.

The controller 101 is an arithmetic device in charge of control that is performed by the server apparatus 100. The controller 101 may be implemented by an arithmetic processing device such as a CPU.

The controller 101 is configured to include three functional modules, namely, a request acquisition unit 1011, a required time estimation unit 1012, and a schedule management unit 1013. Each functional module may be implemented by executing a stored program by the CPU.

The request acquisition unit 1011 determines, based on a request received from the user terminal 300, a medical examination item required for a corresponding user. The medical examination item that is determined at this time is a provisional medical examination item (referred to also as “scheduled medical examination item”) that is estimated from known data.

Furthermore, the request acquisition unit 1011 corrects, based on a request received from the medical terminal 200 operated by a healthcare worker, the scheduled medical examination item for the corresponding user. The medical examination item for the corresponding user is thereby fixed.

The required time estimation unit 1012 creates, based on at least one medical examination item for the user, data (required time information) indicating a time that is required for medical care.

The schedule management unit 1013 creates or updates the medical schedule of the user based on the required time information that is acquired. The determined or updated schedule is transmitted to the user terminal 300.

Details of processes performed by each module will be described later.

A storage 102 is configured to include a main memory and an auxiliary memory. The main memory is a memory where programs to be executed by the controller 101 and data to be used by the control programs are developed. The auxiliary memory is a device where the programs to be executed by the controller 101 and the data to be used by the control programs are stored.

The storage 102 further stores medical examination item data 102A, required time data 102B, and medical schedule data 102C.

The medical examination item data 102A is data for determining, before provision of medical care, the medical examination item necessary for a certain user. The medical examination item data 102A includes at least one of (1) medical record data of the user, and (2) data associating a subjective symptom and a medical examination item (hereinafter “symptom data”).

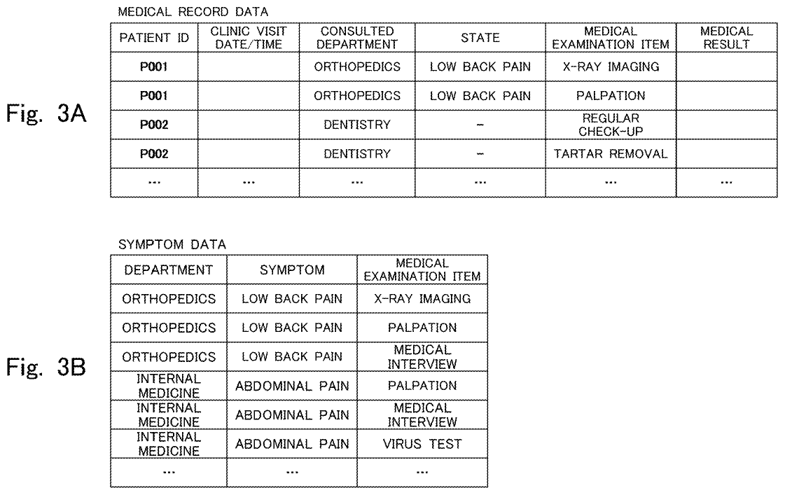

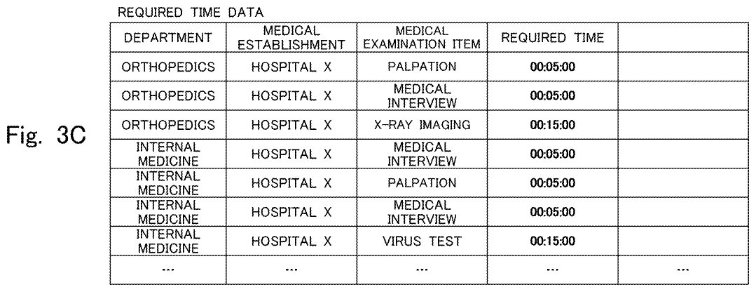

FIG. 3A is an example of the medical record data. For example, the medical record data is data associating, with an identifier (a patient ID) of a user, a plurality of items such as a clinic visit date/time, a consulted department, a state at the time of consultation, a medical examination item, and a medical care result. The medical record data is created by a healthcare worker who provided medical care to the user. By referring to the medical record data, the medical examination item necessary for a user with a past medical record may be determined.

FIG. 3B is an example of the symptom data. The symptom data is data associating a subjective symptom and a recommended medical examination item. By referring to the symptom data, a medical examination item necessary for a user with a subjective symptom may be determined.

The required time data 102B is data defining, for each medical examination item, a time that is required for medical care.

FIG. 3C is an example of the required time data. The required time data is data associating a department, a medical establishment, and a medical examination item with a standard required time for medical care.

By referring to the required time data 102B, a time taken by a predetermined medical examination item may be estimated.

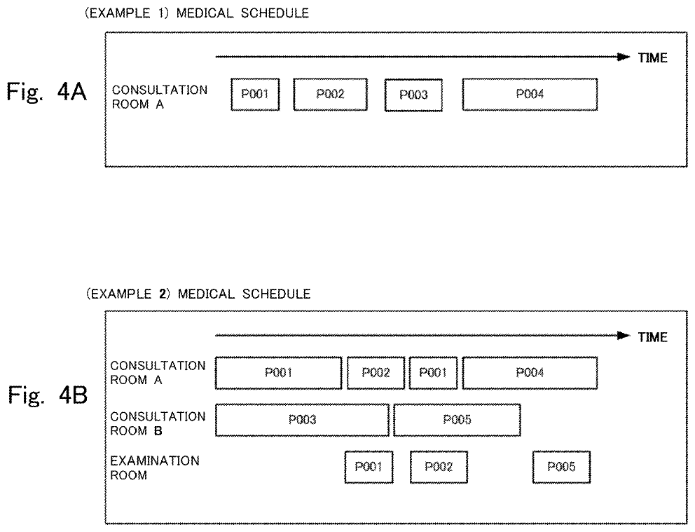

The medical schedule data 102C is data representing a schedule of medical care for a plurality of users. FIGS. 4A and 4B are examples of the medical schedule. In the medical schedule, as indicated by Example 1, a plurality of users may be assigned to one facility (such as a consultation room), or as indicated by Example 2, a plurality of users may be assigned to a plurality of facilities, for example.

A communication unit 103 is a wireless communication interface for connecting the server apparatus 100 to the medical terminal 200 and the user terminal 300. The server apparatus 100 is configured to be capable of communicating with each terminal by a wireless LAN or a mobile communication service such as 3G, LTE, or 5G, for example.

Next, a description will be given of the medical terminal 200.

The medical terminal 200 is a computer such as a personal computer, a tablet computer, a personal digital assistant, a smartphone, a mobile phone, or a wearable computer, for example.

The medical terminal 200 is configured to include a controller 201, a storage 202, a communication unit 203, and an input/output unit 204.

The controller 201 is an arithmetic device in charge of control that is performed by the medical terminal 200. The controller 201 may be implemented by an arithmetic processing device such as a central processing unit (CPU).

Each function described later may be implemented by executing a program stored in the storage 202 described later by the CPU.

The controller 201 implements a function of accessing and interacting with the server apparatus 100. This function may be implemented by a web browser operating on the medical terminal 200. A specific method will be described later.

The storage 202 is configured to include a main memory and an auxiliary memory. The main memory is a memory where programs to be executed by the controller 201 and data to be used by the control programs are developed. The auxiliary memory is a device where the programs (such as a web browser) to be executed by the controller 201 and the data to be used by the control programs are stored. Programs to be executed by the controller 201 may be packaged as an application and stored in the auxiliary memory. Furthermore, an operating system for executing such applications may also be stored. Processes described later are performed by the programs stored in the auxiliary memory being loaded into the main memory and executed by the controller 201.

The main memory may include a random access memory (RAM), and a read only memory (ROM). Furthermore, the auxiliary memory may include an erasable programmable ROM (EPROM), and a hard disk drive (HDD). The auxiliary memory may further include a removable medium, or in other words, a removable recording medium. For example, the removable medium is a universal serial bus (USB) memory, or a disk recording medium such as a compact disc (CD) or a digital versatile disc (DVD).

The communication unit 203 is a communication interface similar to the communication unit 103. For example, the communication unit 203 is capable of communicating with the server apparatus 100 over a wide area network such as the Internet.

The input/output unit 204 is a unit that receives an input operation performed by a user and that presents information to the user. In the present embodiment, it is configured as one touch panel display. That is, a liquid crystal display and control means thereof, or a touch panel and control means thereof are included. The input/output unit 204 may also include an input device such as a keyboard, and a pointing device such as a mouse.

The user terminal 300 is a small computer such as a smartphone, a mobile phone, a tablet computer, a personal digital assistant, a personal computer, or a wearable computer (such as a smartwatch), for example.

The user terminal 300 is configured to include a controller 301, a storage 302, a communication unit 303, and an input/output unit 304.

Additionally, the controller 301, the storage 302, the communication unit 303, and the input/output unit 304 have configurations similar to those of the controller 201, the storage 202, the communication unit 203, and the input/output unit 204, respectively, and a detailed description thereof will be omitted.

Additionally, the configuration illustrated in FIG. 2 is merely an example, and one or some or all of the functions illustrated may be implemented using a dedicated circuit. Furthermore, programs may be stored and executed by a combination of a main memory and an auxiliary memory other than those illustrated.

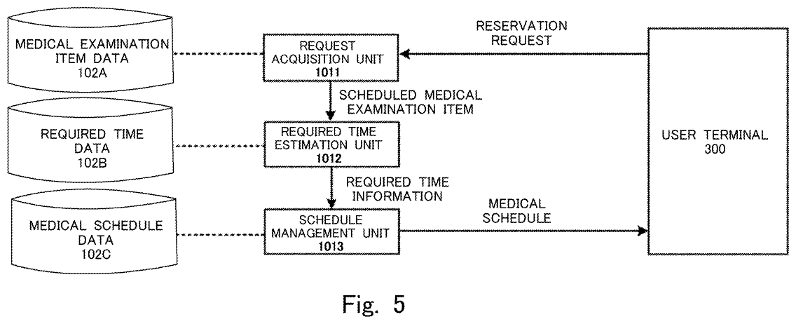

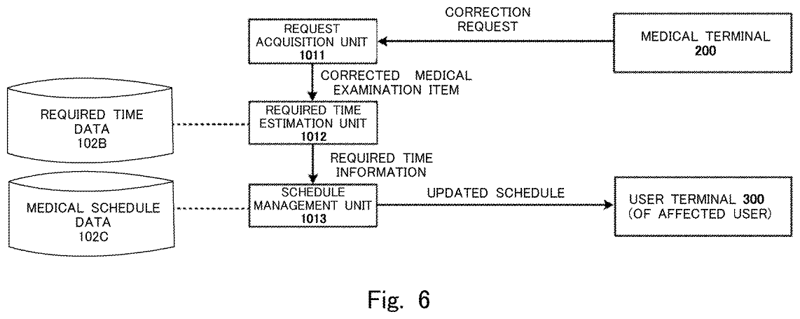

Next, roles of the three functional modules of the server apparatus 100 (the controller 101) will be described with reference to FIGS. 5 and 6 that are diagrams illustrating data transmitted and received among the modules.

FIG. 5 is a diagram illustrating a process, performed by the server apparatus 100, of receiving a request for reservation for medical care (hereinafter “reservation request”) from the user terminal 300, and creating a medical schedule (hereinafter, such a process will be referred to as “first phase”).

In the first phase, the request acquisition unit 1011 receives a reservation request from the user terminal 300 that is associated with a user, and determines, based on the reservation request, the medical examination item that is estimated to be necessary for the corresponding user. The medical examination item is a possible item of medical care, such as a medical interview with a doctor, palpation by a doctor, measurement of blood pressure, a blood test, an electrocardiogram test, an X-ray test, treatment, or medication guidance, for example. There may be two or more medical examination items.

The medical examination item that is estimated to be necessary for a certain user may be acquired based on the medical examination item data 102A described above. The determination result is transmitted to the required time estimation unit 1012.

The required time estimation unit 1012 creates data (the required time information) indicating a time that is required for medical care (a first required time), based on at least one medical examination item for the user. An approximate time taken by each medical examination item may be calculated using the required time data 102B described above. The required time information that is created is transmitted to the schedule management unit 1013.

The schedule management unit 1013 creates the medical schedule based on the required time information that is acquired, by assigning the user on a timeline in the manner illustrated in FIGS. 4A and 4B. Additionally, in the case where there are a plurality of facilities, the user may be assigned to a plurality of facilities (for example, in the case of X-ray imaging, the user is assigned to an X-ray room). Such a process may be performed based on data associating a medical examination item and a facility, for example.

The medical schedule that is created is stored as the medical schedule data 102C. Furthermore, the schedule management unit 1013 transmits the medical schedule that is determined to the user terminal 300.

In the process described above, the medical schedule for a plurality of users is created based on estimated required times. However, the medical schedule is created simply based on past medical records and subjective symptoms felt by the users, and is not always accurate. Accordingly, the server apparatus 100 according to the present embodiment updates the medical schedule by correcting the scheduled medical examination item and the required time based on an opinion of a healthcare worker actually meeting the user.

FIG. 6 is a diagram illustrating a process of updating the medical schedule based on data transmitted from the medical terminal 200 (hereinafter, such a process will be referred to as “second phase”).

Here, the medical terminal 200 is a terminal that is used by a healthcare worker who is in face-to-face contact with the user.

In the second phase, the request acquisition unit 1011 receives a correction request from the medical terminal 200. The correction request is a request to correct the medical examination item that is determined in the first phase. A healthcare worker decides whether the medical examination item determined in the first phase is appropriate, based on a result of observing the user or having a medical interview with the user, for example. If, as a result, the medical examination item is decided to be not appropriate, the healthcare worker transmits the correction request via the medical terminal 200. The correction request includes the medical examination item after correction. The medical examination item after the correction is transmitted to the required time estimation unit 1012.

Next, the required time estimation unit 1012 acquires a required time (a second required time) based on the medical examination item after the correction, by a same method as in the first phase, and creates the required time information. The first required time is updated to the second required time by this process.

The required time information is transmitted to the schedule management unit 1013.

Next, the schedule management unit 1013 updates the medical schedule of the user based on the required time information that is acquired. For example, when a time taken to provide medical care to a user is shortened, the schedule of a following user may be moved forward. Furthermore, when a time taken to provide medical care to a user is increased, the schedule of a following user may be moved backward. That is, schedule is adjusted for a target user and other users who are affected by the user.

The schedule after update is transmitted to the user terminal 300 that is associated with a user who is affected by the update. A user who is affected is a user for whom the medical schedule (a start time or an end time) is changed. Accordingly, that the medical care will be provided earlier than scheduled or that the medical care will be delayed from schedule may be notified, for example.

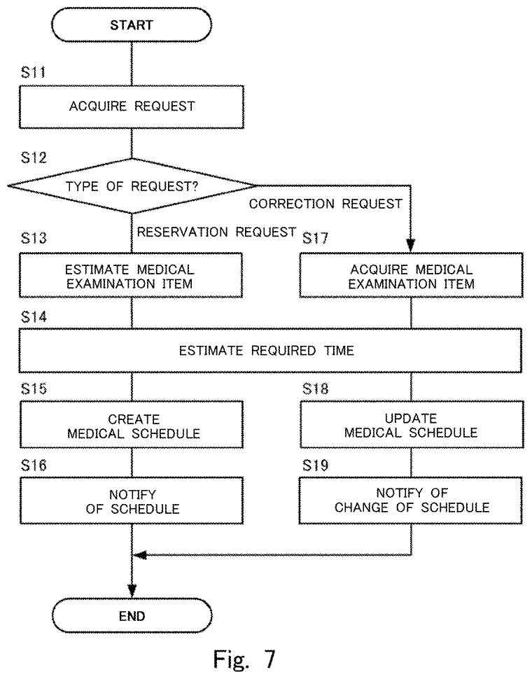

FIG. 7 is a flowchart of a process that is performed by the server apparatus 100 (the controller 101).

First, in step S11, the request acquisition unit 1011 receives a request that is transmitted from the medical terminal 200 or the user terminal 300.

Next, the request acquisition unit 1011 determines whether the received request is a reservation request or a correction request (step S12). In the case where the received request is a reservation request, the process proceeds to step S13.

In step S13, the request acquisition unit 1011 identifies the user who transmitted the reservation request, and determines the medical examination item that is estimated to be necessary. As described above, the medical examination item that is estimated to be necessary may be determined based on past medical records.

Furthermore, in the case where the reservation request transmitted from the user terminal 300 includes answers to a preliminary medical interview (hereinafter “medical interview sheet data”), the medical examination item that is necessary may be determined based on the subjective symptom or the like. Additionally, to conduct a preliminary medical interview, the server apparatus 100 may transmit contents of a medical interview sheet to the user terminal 300, and the user may input the answers via a screen or the like.

In step S14, the required time estimation unit 1012 estimates the time required for medical care, based on the scheduled medical examination item determined in step S13 and the required time data 102B, and creates the required time information.

In step S15, the schedule management unit 1013 creates the medical schedule based on the required time information.

The medical schedule that is created is transmitted, in step S16, to the user terminal 300 that transmitted the reservation request.

In the case where the request received by the request acquisition unit 1011 is a correction request, the process proceeds to step S17.

In step S17, the request acquisition unit 1011 acquires the medical examination item included in the correction request (that is, the medical examination item decided by a healthcare worker to be necessary).

Next, in step S14, the required time estimation unit 1012 estimates the time required for medical care by the same method as the above-described method, and creates the required time information.

In step S18, the schedule management unit 1013 updates the medical schedule based on the required time information.

Then, in step S19, a notification indicating that the medical schedule is changed is transmitted to the user terminal 300 related to a user that is affected by the change in the schedule.

As described above, with the information processing system according to the first embodiment, in the case where the medical examination item that is predicted in advance is different from the medical examination item that is decided by a healthcare worker to be necessary, the required time for medical care may be corrected. This enables an end time of medical care to be accurately calculated. Furthermore, the following medical schedule may be swiftly adjusted or re-created, and convenience of other users may be increased.

### Modification of First Embodiment

In the first embodiment, an embodiment is described where a user makes a reservation for medical care in advance by using the user terminal 300, but reservation is not essential. For example, data requesting medical care (a medical care request) may be transmitted to the server apparatus 100 after the user visits the medical establishment. The medical care request may be transmitted from the user terminal 300 or from the medical terminal 200 (for users) installed at the medical establishment. Furthermore, at this time, the user may answer the medical interview sheet using the user terminal 300 or the medical terminal 200, and the medical care request may be transmitted to the server apparatus 100, together with the contents (the medical interview sheet data).

In this case, the server apparatus 100 may acquire the scheduled medical examination item by using the transmitted medical interview sheet data. Furthermore, a medical schedule that is created may be transmitted to the medical terminal 200 or the user terminal 300.

### Second Embodiment

In the first embodiment, information about the medical schedule is transmitted only to the user terminal 300. In contrast, a second embodiment is an embodiment according to which information about the medical schedule is transmitted to an apparatus that manages vehicles for picking up and dropping off users. This enables a vehicle which a user is to get on to be dispatched to the medical establishment in a timely manner according to an end time of medical care.

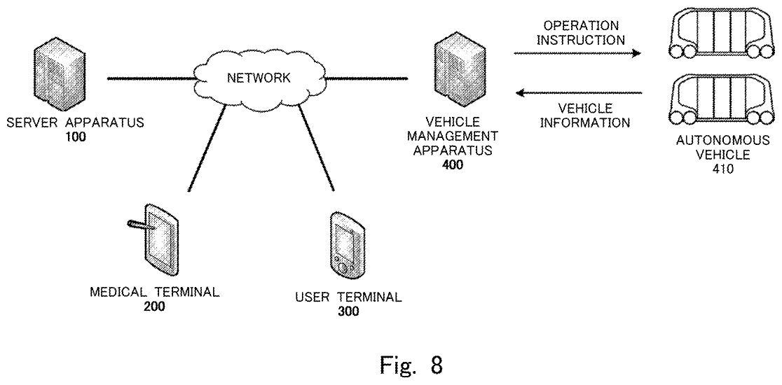

As illustrated in FIG. 8, an information processing system according to the second embodiment is configured to further include a vehicle management apparatus 400, and a plurality of autonomous vehicles 410.

The vehicle management apparatus 400 is an apparatus that manages the plurality of autonomous vehicles 410. The autonomous vehicle 410 is a vehicle that performs unmanned traveling along a predetermined route based on an instruction received from the vehicle management apparatus 400.

The vehicle management apparatus 400 collects, from the plurality of autonomous vehicles 410 that are being managed, information about each vehicle (vehicle information; for example, position information and a status of the vehicle), and also, transmits data instructing operation (an operation instruction) to the plurality of autonomous vehicles 410 that are being managed. The autonomous vehicle 410 may thus be caused to operate along a specified route.

The operation instruction may include data specifying a travel route, a route point, and a destination. Furthermore, the operation instruction may include data specifying a process that is to be performed along the route. A process to be performed along a route, for example, may be parking in a parking lot, calling-out to a user, pick-up and drop-off of a user, and the like.

In the second embodiment, when a medical schedule is created or updated, the schedule management unit 1013 creates information (reservation information) including an identifier of the user, an identifier of the medical establishment, a time when medical care ends, and the like, and transmits the same to the vehicle management apparatus 400. Furthermore, the vehicle management apparatus 400 creates an operation instruction for the autonomous vehicle 410 based on the reservation information, and transmits the same. Moreover, the autonomous vehicle 410 performs a task of picking up and dropping off the user based on the received information.

According to such an embodiment, a vehicle for picking up and dropping off a user may be automatically dispatched based on the end time of medical care.

Additionally, in the present embodiment, the autonomous vehicle is cited as an example, but a vehicle that a user is to get on may alternatively be a manned vehicle (such as a taxi).

### Modification of Second Embodiment

In the second embodiment, the server apparatus 100 transmits information about the medical schedule to the apparatus that manages the vehicles that the users are to get on, but the server apparatus 100 may alternatively transmit the information about the medical schedule to other apparatuses. For example, the information about the medical schedule may be transmitted to an apparatus that is associated with a dispensing pharmacy that is used by the user after medical care. In this case, computerized prescription data may be transmitted at the same time. This enables dispensing of medicine to be started in a timely manner according to the end time of medical care.

Furthermore, the information about the medical schedule may be transmitted to both the apparatus that is associated with a dispensing pharmacy and the vehicle management apparatus. This enables the autonomous vehicle to perform two types of tasks, namely, “picking up of medicine” and “picking-up and dropping-off of user”, for example.

### Modification

The embodiments described above are merely examples, and the present disclosure may be implemented with changes as appropriate within the scope of the disclosure.

For example, processes and means described in the present disclosure may be freely combined and implemented to the extent that no technical conflict exists.

A process that is described to be performed by one apparatus may be shared and performed by a plurality of apparatuses. Furthermore, processes described to be performed by different apparatuses may be performed by one apparatus. Which function is to be implemented by which hardware configuration (server configuration) in a computer system may be flexibly changed.

The present disclosure may also be implemented by supplying computer programs for implementing the functions described in the embodiments described above to a computer, and by one or more processors of the computer reading out and executing the programs. Such computer programs may be provided to the computer by a non-transitory computer-readable storage medium that can be connected to a system bus of the computer, or may be provided to the computer via a network. The non-transitory computer-readable storage medium may include, for example, any type of disk including magnetic disks (floppy (registered trademark) disks, hard disk drives (HDDs), etc.) and optical disks (CD-ROMs, DVD discs, Blu-ray discs, etc.), read-only memories (ROMs), random access memories (RAMs), EPROMs, EEPROMs, magnetic cards, flash memories, optical cards, and any type of medium suitable for storing electronic instructions.

What is claimed is:

1. An information processing apparatus for estimating a required time for medical care in relation to a user visiting a medical establishment, the information processing apparatus comprising a controller configured to:
determine a scheduled medical examination item as at least one medical examination item that is scheduled for the user;
calculate a first required time based on the scheduled medical examination item;
receive a correction of the scheduled medical examination item while the user is receiving medical care; and
calculate a second required time based on the scheduled medical examination item after the correction.

2. The information processing apparatus according to claim 1, further comprising a storage configured to store required time data that is data associating a required time with each medical examination item, wherein
the controller calculates the first required time and the second required time based on the required time data.

3. The information processing apparatus according to claim 1, wherein the controller determines the scheduled medical examination item based on information that is obtained from a result of a preliminary medical interview with the user.

4. The information processing apparatus according to claim 1, wherein the controller determines the scheduled medical examination item based on a medical record of the user.

5. The information processing apparatus according to claim 1, wherein the controller corrects the scheduled medical examination item based on information that is acquired from a healthcare worker who is providing medical care to the user.

6. The information processing apparatus according to claim 1, wherein the controller creates a medical schedule for a plurality of users based on the required time for medical care estimated for the plurality of users.

7. The information processing apparatus according to claim 6, wherein, in a case where the second required time is calculated for a first user, the controller adjusts the medical schedule for a second user who is to receive medical care after the first user.

8. The information processing apparatus according to claim 6, wherein the controller estimates, based on the medical schedule, a time of end of medical care for a predetermined user.

9. The information processing apparatus according to claim 8, wherein the controller transmits the estimated time to an apparatus related to the user.

10. The information processing apparatus according to claim 9, wherein the apparatus related to the user is a first apparatus that manages a mobile body that the user gets on after receiving medical care.

11. The information processing apparatus according to claim 10, wherein the controller transmits, to the first apparatus, a route that passes through a pharmacy that provides medicine to the user and the medical establishment where the user is receiving medical care.

12. The information processing apparatus according to claim 9, wherein the apparatus related to the user is a second apparatus that is associated with a pharmacy that provides medicine to the user.

13. An information processing system comprising a first apparatus configured to estimate a required time for medical care in relation to a user visiting a medical establishment, and a second apparatus that is associated with the user, wherein
the first apparatus includes a controller configured to:
determine a scheduled medical examination item as at least one medical examination item that is scheduled for the user;
calculate a first required time based on the scheduled medical examination item;
receive a correction of the scheduled medical examination item while the user is receiving medical care;
calculate a second required time based on the scheduled medical examination item after the correction;
create a medical schedule for a plurality of the users based on the first required time and the second required time; and
transmit a predetermined notification to the second apparatus based on the medical schedule.

14. The information processing system according to claim 13, wherein the controller estimates, based on the medical schedule, a time of end of medical care for a predetermined user.

15. The information processing system according to claim 14, wherein the controller transmits the estimated time to an apparatus related to the user.

16. The information processing system according to claim 15, wherein the apparatus related to the user is an apparatus that manages a mobile body that the user gets on after receiving medical care.

17. The information processing system according to claim 16, wherein the controller transmits, to the apparatus that manages the mobile body, a route that passes through a pharmacy that provides medicine to the user and the medical establishment where the user is receiving medical care.

18. The information processing system according to claim 15, wherein the apparatus related to the user is an apparatus that is associated with a pharmacy that provides medicine to the user.

19. A non-transitory storage medium storing a program for causing an information processing apparatus for estimating a required time for medical care in relation to a user visiting a medical establishment to:
determine a scheduled medical examination item as at least one medical examination item that is scheduled for the user;
calculate a first required time based on the scheduled medical examination item;
receive a correction of the scheduled medical examination item while the user is receiving medical care; and
calculate a second required time based on the scheduled medical examination item after the correction.

20. The non-transitory storage medium according to claim 19, wherein the program causes the information processing apparatus to perform a process of creating a medical schedule for a plurality of the users based on the required time for medical care estimated for the plurality of the users.
